(19) United States
(12) Patent Application Publication
(10) Pub. No.: US 2025/0319026 A1
(43) Pub. Date: Oct. 16, 2025

(54) VITAMIN COMPOSITION FOR ORAL ADMINISTRATION

(71) Applicants: Valerii Zaiko, Sunny Isles Beach, FL (US); Dmitrii Donskoi, Sunny Isles Beach, FL (US)

(72) Inventors: Valerii Zaiko, Sunny Isles Beach, FL (US); Dmitrii Donskoi, Sunny Isles Beach, FL (US)

(21) Appl. No.: 18/208,287

(22) Filed: Jun. 11, 2023

(51) Int. Cl. A61K 9/14; A61K 31/4415; A61K 31/51; A61K 31/714; A61K 47/26

(52) U.S. Cl. CPC A61K 9/143; A61K 31/4415; A61K 31/51; A61K 31/714; A61K 47/26

(57) ABSTRACT

A composition of the present invention is used for oral administration. The composition incudes thiamine at least 5% by weight, pyridoxine at least 5 wt. %; cyanocobalamin at least 0.1 wt. %, polysorbate 40 at least 1.8 wt. %, and water for the rest, up to 100 wt. %. The composition provides high concentrations of pyridoxine, thiamine, cyanocobalamin, stability for a long period of storage under standard conditions.

### FIELD OF THE INVENTION

The present invention relates to a method of vitamin preparations, in particular preparation of a liquid pharmaceutical composition containing vitamins B, namely, pyridoxine, thiamine, cyanocobalamin.

### BRIEF DESCRIPTION OF THE INVENTION

Neurotropic vitamins B have a beneficial effect on inflammatory and degenerative diseases of the nerves and musculoskeletal system. These vitamins promote increased blood flow and improve the functioning of the nervous system. Thiamine plays a key role in the metabolism of carbohydrates, as well as in the Krebs cycle, with subsequent participation in the synthesis of TPP (thiamine pyrophosphate) and ATP (adenosine triphosphate).

Pyridoxine is involved in protein metabolism, and in part, in the metabolism of carbohydrates and fats. The physiological function of both vitamins is to potentiate the action of each other, which manifests itself in a positive effect on the nervous, neuromuscular and cardiovascular systems. In vitamin B6 deficiency, widespread deficiencies are quickly reversed after administration of these vitamins. Cyanocobalamin is involved in the synthesis of the myelin sheath, stimulates hematopoiesis, reduces pain associated with lesions of the peripheral nervous system, stimulates nucleic metabolism through the activation of folic acid.

Compositions containing vitamins B, including multivitamins, are well known in the prior art. For example, one of the prior at references, such CN 104606220 A, teaches a composition that includes vitamins D3, E, K1, C, B1, B6, B12, histidine, polysorbate 80. This composition does not provide the amounts of B vitamins necessary for the body.

Another prior art reference, such as CN 104256652 A, teaches a composition that includes nanoliposomes obtained using lecithin, cholesterol, polysorbate-80, this composition contains vitamins A1, D3, E, K, C, B1, B2, B3, B5, B6, B7, B12. This composition also does not provide enough B vitamins.

Another composition is disclosed in CN 102100304 B, includes vitamins, B1, B6, B12, folic acid, calcium pantothenate, glycine, polysorbate-80. This composition can be taken as the closest analogue. The disadvantage of this composition is the inability to provide high concentrations of B vitamins, insufficient storage stability.

One of the major disadvantages and drawbacks of the aforementioned prior art references is the absence of both pyridoxine, thiamine, cyanocobalamin in sufficiently high concentrations to ensure the supply of these vitamins to cover the necessary needs of the body. In experimental studies, it was found that the use of polysorbate-40 as a solvent in an amount of at least 1.8 wt. % makes it possible to bring the concentrations of thiamine and pyridoxine to at least 6 wt. %, and the concentration of cyanocobalamin at least 0.1 wt %. The solution thus obtained is stable and has a long shelf life under standard conditions.

The use of polysorbate-40 makes it possible to manufacture a composition that provides high concentrations of B vitamins, namely B1, B6, B12. It should be noted that a composition is known from the prior art, including vitamin B1 and polysorbate 40 is disclosed in CN 104721190 B and according to patent RU 2421207 C2 it is allowed to manufacture a composition containing vitamins B1, B6, B12, polysorbate 40.

However, the known composition disclosed in CN 104721190 B does not include vitamins B6, B12, and the concentration of vitamin B1 is significantly less than in the composition by the decision of the customer. As further disclosed in prior art reference RU 2421207 C2, a large number of different components are disclosed (along with many others, vitamins B1, B6, B12, polysorbate 40), but no specific example of a composition that simultaneously includes vitamins B1, B6, B12, polysorbate 40, with specific concentrations of components, i.e. in fact, a composition comprising all of these components simultaneously has not been disclosed.

None of the prior art references known today teaches or at least suggests the composition with high concentrations of pyridoxine, thiamine, cyanocobalamin, stability for a long period of storage under standard conditions.

### SUMMARY OF THE INVENTION

A composition for oral administration includes at least 5% of thiamine, at least 5% of pyridoxine, at least 0.1% of cyanocobalamin, at least 1.8% of polysorbate 40, and 88.1% of fluid.

An advantage of the present invention is to provide the composition provides high concentrations of pyridoxine, thiamine, cyanocobalamin, stability for a long period of storage under standard conditions.

Obviously, both the above general description and the following detailed description are only given as an example and an explanation and are not the limitations of this utility model.

The objects and advantages of the present invention will be more readily apparent from inspection of the following specification, taken in connection with the accompanying drawing, wherein like numerals refer to like parts throughout and in which an embodiment of the present invention is described and illustrated.

The exact manner in which the foregoing and other objects and advantages of the invention are achieved in practice will become more clearly apparent when reference is made to the following detailed description of the preferred embodiments of the invention described in detail in the following specification and shown in the accompanying drawings, where in like reference numbers indicate corresponding parts throughout.

### DETAILED DESCRIPTION OF THE INVENTION

As used herein, the singular forms “a,” “an,” and “the” include plural referents unless the context clearly dictates otherwise. Thus, for example, the term “module” is intended to mean one or more modules or a combination of modules. Furthermore, as used herein, the term “based on” includes based at least in part on. Thus, a feature that is described as based on some cause, can be based only on that cause, or based on that cause and on one or more other causes.

It will be apparent that multiple embodiments of this disclosure may be practiced without some or all of these specific details. In other instances, well-known process operations have not been described in detail in order not to unnecessarily obscure the present embodiments. The following description of embodiments includes references to the accompanying drawing. The drawing shows illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Alluding to the above, for purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

An inventive composition for oral administration includes thiamine, pyriodxin, cyanocobalamin, including: thiamine at least 5 wt. %, pyridoxine at least 5 wt. %, cyanocobalamin at least 0.1 wt. %, polysorbate 40 at least 1.8 wt. %, water the rest, up to 100 wt. %. The composition may be presented as a solution for oral administration.

The composition can also be presented as a solution adsorbed by stirring in a dispersant on nanoparticles of amorphous mesoporous silicon dioxide with a mesopore size of 0.2 to 1.8 nm. The solution for oral administration can be prepared, for example, as follows: thiamine 9% by weight; pyridoxine 9 wt. %; cyanocobalamin 0.2 wt. %; polysorbate-40 1.8 wt. %; water 80 wt. %.

The solution for oral administration can also be prepared, for example, as follows: thiamine 5 wt. %; pyridoxine 5 wt %; cyanocobalamin 0.16% by weight; polysorbate 40 37.84% by weight; water 52 wt. %.

Data from experimental studies have shown that the use of polysorbate 40 as a solvent in an amount of at least 1.8 wt. % makes it possible to obtain significantly higher than known concentrations of thiamine and pyridoxine, at least 6 wt. %, and the concentration of cyanocobalamin at least 0.1 wt. %. The solution thus obtained is stable and has a long shelf life under standard conditions.

Such compositions that provide sufficiently high concentrations of pyridoxine, thiamine, cyanocobalamin are not known from the prior art. The technical result is to obtain a composition that includes B vitamins (pyridoxine, thiamine, cyanocobalamin), which meets several conditions: firstly, it contains high concentrations of pyridoxine, thiamine, cyanocobalamin, and secondly, it is stable for a long shelf life at standard conditions, thirdly, does not require the content of any additional excipients.

A composition that would meet the above three conditions has not previously been obtained. The following are examples of the possible preparation of the developed composition.

In preparation of the composition, a reactor was charged with 6 g of pyridoxine, 6 g of thiamine, and 0.158 g of cyanocobalamin. Then 50 g of distilled water was added and stirred until complete dissolution at a temperature of 45-50° C. Then, 37.842 g of polysorbate 40 was introduced in small portions. Stirred for 1 hour at room temperature. The output was a stable transparent red solution. With repeated cooling to +3° C., precipitation was not observed.

Alternatively, the reactor was charged with 7 g of pyridoxine, 7 g of thiamine, and 0.183 g of cyanocobalamin. Then, it was added 60 g of distilled water and stirred until complete dissolution at a temperature of 45-50°° C. Then, 25.817 g of polysorbate 40 were introduced in small portions. Stirred for 1 hour at room temperature. The output was a stable transparent red solution.

Alternatively, the reactor was loaded with 8 g of pyridoxine, 8 g of thiamine and 0.194 g of cyanocobalamin. Then, it was added 70 g of distilled water and stirred until complete dissolution at a temperature of 45-50° C. Then, 13.806 g of polysorbate 40 was introduced in small portions. Stirred for 1 hour at room temperature. The output was a stable transparent red solution.

Alluding to the above, alternatively, the reactor was loaded with 9 g of pyridoxine, 9 g of thiamine and 0.198 g of cyanocobalamin. Then, 80 g of distilled water was added and stirred until complete dissolution at a temperature of 45-50° C. Then, 1.802 g of polysorbate 40 was introduced in small portions. Stirred for 1 hour at room temperature. The output was a stable transparent red solution.

Still in another alternative embodiment, the reactor was loaded with 6 g of pyridoxine, 6 g of thiamine and 0.1 g of cyanocobalamin. Then, 45 g of distilled water was added and stirred until complete dissolution at a temperature of 45-50° C. Then, 42.9 g of polysorbate 40 were introduced in small portions. Stirred for 1 hour at room temperature. The output was a stable transparent red solution. With repeated cooling to +3° C., precipitation was not observed.

Furthermore, the reactor was loaded with 5 g of pyridoxine, 5 g of thiamine and 0.16 g of cyanocobalamin. Then, 52 g of distilled water was added and stirred until complete dissolution at a temperature of 45-50° C. Then, 37.84 g of polysorbate 40 was introduced in small portions. Stirred for 1 hour at room temperature. The output was a stable transparent red solution. With repeated cooling to +3° C., precipitation was not observed.

Alternatively, he reactor was loaded with 8 g of pyridoxine, 9 g of thiamine and 0.2 g of cyanocobalamin. Them, 81 g of distilled water was added and stirred until complete dissolution at a temperature of 45-50° C. Then 1.8 g of polysorbate 40 was introduced in small portions. Stirred for 1 hour at room temperature. The output was a stable transparent red solution.

The physical and chemical properties of the resulting solution were checked during the first year of storage—every 3 months, during the second and third years of storage—every 6 months, the solution is stable, during the tests carried out with repeated cooling to +3° Since the precipitation was not observed.

The shelf life of the obtained solutions in sealed conditions in a place protected from light at a temperature of 10 to 25° C. for three years.

A composition in the form of a solution deposited on amorphous silica particles can be obtained as follows. The required amount of pyridoxine, thiamine, cyanocobalamin is dissolved in the required amount of distilled water when heated to a temperature of 80-90° C., stirring at a speed of about 1000 rpm to about 2000 rpm, then cooling is carried out to a temperature of below 60° C. to below 45° C., adding amorphous mesoporous silica with a mesopore size of 0.2 to 1.8 nm, stirred at a speed of about 1000 rpm to about 2000 rpm for about 0.5 h, then increase mixing speed up to 7000-9000 rpm until a homogeneous product in the form of a free-flowing powder is obtained.

The following are examples of obtaining a composition in the form of a solution deposited on particles of amorphous silicon dioxide.

Alternatively, 7 g of pyridoxine, 7 g of thiamine, 0.183 g of cyanocobalamin were dissolved in 60 g of distilled water with constant stirring at 1500 rpm and heating to 80° C., with the addition of polysorbate 40 in small portions. The resulting solution was cooled to a temperature below 55° C. and added to amorphous silicon dioxide with mesopores from 0.2 to 1.8 nm in an amount of 10 g with stirring at 1000 rpm in an IKA T 50 digital disperser for 0.5 h. Then the speed was increased to 7000 rpm min to improve the dispersibility of reactants. A free-flowing reddish powder was obtained.

Alternatively, 6 g of pyridoxine, 6 g of thiamine, 0.16 g of cyanocobalamin were dissolved in 50 g of distilled water and dissolved with constant stirring at 2000 rpm and heating to 85° C. with the addition of polysorbate 40 in small portions. The resulting solution was cooled to a temperature below 60° C. and added to amorphous silicon dioxide with mesopores from 0.2 to 1.8 nm in an amount of 15 g with stirring in an IKA T 50 digital disperser at 2000 rpm for 0.5 h. Then the speed was increased up to 8000 rpm to increase the dispersibility and uniformity of the distribution of substances. A free flowing reddish powder was obtained.

Alternatively, 8 g of pyridoxine, 9 g of thiamine, 0.2 g of cyanocobalamin were dissolved in 81 g of distilled water with stirring at 2000 rpm and heated to 90° C. while adding polysorbate 40 in small portions. The resulting solution was cooled to a temperature below 45° C. and added to 5 g of amorphous silicon dioxide with mesopores from 0.2 to 1.8 nm with vigorous stirring at 1500 rpm in an IKA T 50 digital disperser for 0.5 h. Then the speed was increased to 9000 rpm to improve the dispersibility of reactants. A free flowing reddish powder was obtained.

Checking the physicochemical properties of the resulting drug was carried out during the first year of storage—every 3 months, during the second and third years of storage—every 6 months, the drug is stable.

The shelf life of the resulting drug in sealed conditions in a place protected from light at a temperature of 5 to 25° C. for three years.

Thus, in relation to the resulting composition, it is necessary to draw the following conclusions: solutions of B vitamins with the addition of an emulsifier, stable over a wide temperature range; this dosage form can serve as an effective alternative to existing analogues for oral administration; in dry dosage forms declared on the market of the pharmaceutical industry there is no cyanocobalamin. This is due to the extreme photo and thermal lability of this molecule. By obtaining the proposed composition, this issue is resolved.

A composition has been obtained that includes B vitamins (pyridoxine, thiamine, cyanocobalamin), which, firstly, contains their high concentrations, providing the amounts of these vitamins corresponding to the needs of the body, and secondly, is stable for a long period of storage when standard conditions, thirdly, does not require the content of any additional excipients.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A composition for oral administration, comprising:
at least 5% of thiamine;
at least 5% of pyridoxine;
at least 0.1% of cyanocobalamin;
at least 1.8% of polysorbate 40; and
88.1% of fluid.

2. The composition as set forth in claim 1, wherein the fluid is further defined by a water.

3. The composition as set forth in claim 1, wherein the composition is adsorbed by mixing in a dispersant on nanoparticles of amorphous mesoporous silicon dioxide with a mesopore size of 0.2 to 1.8 nm.

4. The composition as set forth in claim 1, wherein the composition includes 9% of thiamine, 9% of pyridoxine, 0.2% of cyanocobalamin, 1.8% of polysorbate 40, and water 80 wt. %.0.5.

5. The composition as set forth in claim 1, wherein the composition includes 5% of thiamine, 5% of pyridoxine, 0.16% of cyanocobalamin, 37.84% of polysorbate 40, and 52% of water.

6. A method of fabrication a composition for oral administration, said method comprising the steps of:
mixing at least 5% of thiamine with at least 5% of pyridoxine, at least 0.1% of cyanocobalamin, and at least 1.8% of polysorbate 40;
adding 88.1% of fluid; and
adsorbed the composition by mixing in a dispersant on nanoparticles of amorphous mesoporous silicon dioxide with a mesopore size of 0.2 to 1.8 nm.

7. The method as set forth in claim 6, wherein the step of mixing the composition is further defined by mixing 9% of thiamine, 9% of pyridoxine, 0.2% of cyanocobalamin, 1.8% of polysorbate 40, and water 80 wt. %.0.5.

8. The method as set forth in claim 6, wherein the step of mixing the composition is further defined by mixing 9% of thiamine, 9% of pyridoxine, 0.2% of cyanocobalamin, 1.8% of polysorbate 40, and water 80 wt. %.0.5.

9. The method as set forth in claim 6, wherein the step of mixing the composition is further defined by mixing 5% of thiamine, 5% of pyridoxine, 0.16% of cyanocobalamin, 37.84% of polysorbate 40, and 52% of water.
